(19) United States
(12) Patent Application Publication
(10) Pub. No.: US 2023/0363905 A1
(43) Pub. Date: Nov. 16, 2023

(54) METHODS TO FOLD AND FORM DELIVERY SYSTEM BALLOONS TO REDUCE THE DEPLOYED THV HEIGHT ASYMMETRY

(71) Applicant: EDWARDS LIFESCIENCES CORPORATION, Irvine, CA (US)

(72) Inventors: Ramin Samari, Pasadena, CA (US); Michael C. Murad, Lake Mathews, CA (US); Michael R. Bialas, Lake Forest, CA (US)

(21) Appl. No.: 18/358,855

(22) Filed: Jul. 25, 2023

Related U.S. Application Data
(63) Continuation of application No. PCT/US2022/013807, filed on Jan. 26, 2022.
(60) Provisional application No. 63/142,157, filed on Jan. 27, 2021.

(51) Int. Cl. A61F 2/24; A61M 29/02

(52) U.S. Cl. CPC A61F 2/2433; A61M 29/02; A61M 2025/1004

(57) ABSTRACT

The present embodiments relate generally to devices, systems, and methods for dilating implants utilizing dilation devices. An example includes a dilation device for an implant. The dilation device includes an inflatable body having a proximal end and a distal end, and a central body positioned between the proximal end and the distal end and having a length. The central body is configured to press an inner surface of the implant to dilate the implant. The inflatable body includes at least one outer inflatable body fold radius of curvature greater than or equal to one half a thickness of the inflatable body.

### CROSS-REFERENCE TO RELATED APPLICATIONS

This application is a continuation of PCT patent application no. PCT/US2022/013807 filed on Jan. 26, 2022, which claims the benefit of U.S. Provisional Application No. 63/142,157, filed Jan. 27, 2021, the entire contents of each of which are incorporated herein by this specific reference.

### BACKGROUND OF THE INVENTION

A variety of maladies may affect an individual's body. Such maladies may be of the individual's heart, and may include maladies of the individual's heart valves, including the aortic, mitral, tricuspid, and pulmonary valves. Prosthetic implants, such as prosthetic heart valves, may be provided to replace a portion of a patient's heart. Prosthetic aortic, mitral, tricuspid, and even pulmonary valves may be provided. Implants may be deployed to the desired portion of the patient's body percutaneously, in a minimally invasive manner. Such deployment may occur transcatheter, in which a catheter may be deployed through the vasculature of an individual. During deployment of such implants, the implants must be dilated to provide an expanded configuration for such implant.

Balloon expandable Transcatheter Heart Valves (THV) may sometimes be deployed in an asymmetric shape. This is undesirable due to the fact that the THV might not perform as well when deployed in an asymmetric shape. For example, paravalvular leakage (PVL), reduced durability of the valve, or both PVL and reduced durability of the valve may occur. In addition, the tissues of the THV might not co-apt completely. Accordingly, it may be advantageous to reduce an asymmetric shape in a THV.

### SUMMARY

The present devices, systems, and methods may relate to devices, systems, and methods for dilating implants such as a THV such that the THV has a reduced possibility of being deployed in an asymmetric shape. For example, devices, systems, and methods described herein may include inflatable bodies such as balloons, which may include certain balloon pleats, balloon folds, or both balloon pleats and balloon folds, as well as balloon forms such that the balloon may reduce asymmetries in THV height after deployment of the THV. Such devices, systems, and methods may include inflatable bodies configured to be inflated to dilate the implant to improve the possibility that the inflation of the inflatable body and the deployment of the implant may be symmetrical.

Embodiments herein may include a dilation device for an implant. The dilation device may comprise an inflatable body having a proximal end and a distal end, and a central body positioned between the proximal end and the distal end and having a length. The central body may be configured to press an inner surface of the implant to dilate the implant. The inflatable body may include at least one outer inflatable body fold radius of curvature greater than or equal to one half a thickness of the inflatable body.

Embodiments herein may include a dilation device for an implant. The dilation device may comprise an inflatable body having a proximal end and a distal end, and a central body positioned between the proximal end and the distal end and having a length. The central body may be configured to press an inner surface of the implant to dilate the implant. The inflatable body may include a lubricious exterior surface.

Embodiments herein may include an implant delivery system. The implant delivery system may include a delivery apparatus. The implant delivery system may include an inflatable body coupled to the delivery apparatus and having a proximal end and a distal end, and a central body positioned between the proximal end and the distal end and having a length. The central body may be configured to press an inner surface of the implant to dilate the implant. The inflatable body may include at least one outer inflatable body fold radius of curvature greater than or equal to one half a thickness of the inflatable body.

Embodiments herein may include an implant delivery system. The implant delivery system may include a delivery apparatus. The implant delivery system may include an inflatable body coupled to the delivery apparatus and having a proximal end and a distal end, and a central body positioned between the proximal end and the distal end and having a length. The central body may be configured to press an inner surface of an implant to dilate the implant. The inflatable body may include a lubricious exterior surface.

Embodiments herein may include a method comprising inserting a dilation device for an implant into a body. The method may include dilating the implant utilizing the dilation device. The dilation device may comprise an inflatable body having a proximal end and a distal end, and a central body positioned between the proximal end and the distal end and having a length. The central body may be configured to press an inner surface of the implant to dilate the implant. The inflatable body may include at least one outer inflatable body fold radius of curvature greater than or equal to one half a thickness of the inflatable body.

Embodiments herein may include a method. The method may comprise providing an inflatable body having a proximal end and a distal end, and a central body positioned between the proximal end and the distal end and having a length, the central body configured to press an inner surface of the implant to dilate the implant. The method may comprise rotating the inflatable body along an axis from the proximal end through the distal end while compressing the inflatable body.

Embodiments herein may include a method. The method may include inserting a dilation device for an implant into a body. The method may include dilating the implant utilizing the dilation device. The dilation device may comprise an inflatable body having a proximal end and a distal end, and a central body positioned between the proximal end and the distal end and having a length. The central body may be configured to press an inner surface of the implant to dilate the implant. The inflatable body may be formed with a pleated inside diameter greater than a pleat height.

Embodiments herein may include a method. The method may include inserting a dilation device for an implant into a body. The method may include dilating the implant utilizing the dilation device. The dilation device may comprise an inflatable body having a proximal end and a distal end, and a central body positioned between the proximal end and the distal end and having a length. The central body may be configured to press an inner surface of the implant to dilate the implant, the inflatable body formed using a mandrel positioned within the inflatable body wherein a ratio of a compressed outer diameter of the inflatable body to an outer diameter of the mandrel is 2.5 or larger.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar embodiments.

FIG. 1 is a perspective view of a deployed prosthetic valve.

FIG. 2 is a top view of the prosthetic valve shown in FIG. 1.

FIGS. 3A, 3B, and 3C are views of inflatable body cross sections.

FIG. 4A illustrates a perspective view of a deployed prosthetic valve.

FIG. 4B illustrates a top view of the prosthetic valve shown in FIG. 4A.

FIG. 5 illustrates a view of an inflatable body cross section that may yield high deployed THV height asymmetry.

FIG. 6 illustrates an example outer inflatable body fold radius of curvature.

FIG. 7A illustrates a cross sectional view of a relationship between a pleated inner diameter and a pleat height.

FIG. 7B illustrates a cross sectional view of a relationship between a mandrel outer diameter and a compressed outer diameter.

FIG. 8A illustrates a cross sectional view of a pleated inflatable body.

FIG. 8B illustrates a cross sectional view of a pleated inflatable body relative to a compression tool.

FIG. 8C illustrates a cross sectional view of a compressed inflatable body.

FIG. 9A illustrates a cross sectional view of rotation of an inflatable body.

FIG. 9B illustrates a cross sectional view of a compressed inflatable body.

FIG. 10 illustrates a side view of a delivery apparatus according to an embodiment of the present disclosure.

FIG. 11 illustrates a detail view of a portion of the delivery apparatus shown in FIG. 10 according to an embodiment of the present disclosure.

### DETAILED DESCRIPTION

The following description and examples illustrate some example embodiments of the disclosure in detail. Those of skill in the art will recognize that there are numerous variations and modifications of the disclosure that are encompassed by its scope. Accordingly, the description of a certain example embodiment should not be deemed to limit the scope of the present disclosure.

The systems and methods described herein may generally relate to devices, systems, and methods for dilating implants such as a Transcatheter Heart Valve (THV) such that the THV has a reduced possibility of being deployed in an asymmetric shape. For example, systems and methods described herein may relate to inflatable bodies such as balloons, which may include certain balloon pleats, balloon folds, or both balloon pleats and balloon folds and form shapes that may reduce or eliminate asymmetries in THV height after deployment of the THV. Such devices, systems, and methods may include inflatable bodies configured to be inflated to dilate the implant where the inflation may be symmetrical.

Various methods may be employed to reduce an asymmetric shape when deployed. In one example, an outer inflatable body fold radius of curvature may be greater than or equal to one half a thickness of the inflatable body. In embodiments, the inflatable body may include a lubricious exterior surface. For example, a lubricious coating may be provided on the exterior surface of the inflatable body. The lubricious exterior surface may be a surface thermally bonded to the inflatable body. In another example, the lubricious exterior surface may comprise a textured surface. The textured surface may reduce the surface friction on the inflatable body exterior surface, e.g., reduce the contact surface area between the exterior surface of the inflatable body and itself or with the THV. In embodiments, the inflatable body may be rotated along an axis from a proximal end through a distal end of the inflatable body while compressing the inflatable body. A rotation of the inflatable body during a compression operation of the inflatable body may be used to eliminate or reduce pleat hooks from forming. In some aspects, one or more of the various methods or other features discussed above may be combined to reduce the formation of an asymmetric shape when the inflatable body is deployed. In an aspect, the lubricious exterior surface of the inflatable body may be a bead blasted surface or other reduced friction surface or reduced friction coating. Bead blasting may include applying small particles such as quartz sand or small spherical particles of glass or other small particles to the exterior surface of the inflatable body. Such application may be a high pressure application of small particles.

FIG. 1 illustrates a perspective view of an implant 10 in the form of a prosthetic valve. The prosthetic valve, in embodiments, may comprise a Transcatheter Heart Valve (THV) as represented in FIG. 1, or may comprise another form of prosthetic valve in embodiments as desired. In embodiments, other forms of implants may be utilized as desired. The implant 10, as shown, may have an asymmetric shape. As discussed above, the asymmetric shape may be undesirable. The configuration shown in FIG. 1 may represent an undesirable deployment of the implant 10.

The implant 10 may include a body 12 that may have a proximal end 14, a distal end 16, and a height between the proximal end 14 and the distal end 16. The proximal end 14 may comprise an outflow end of the implant 10, and the distal end 16 may comprise an inflow end of the implant 10.

The body 12 may comprise a valve body that may include a frame 20 that may include a plurality of struts 22 that may join together at junctures 24 and may have spaces 26 between the struts 22. The spaces 26 may comprise openings of the frame 20 that may allow fluid flow therethrough or the passage of other components therethrough. The configuration of the frame may vary in other embodiments.

The frame 20 may be configured to allow the body 12 to be collapsible and expandable, with the frame 20 being crimped to move to a collapsed (or undeployed or unexpanded) state and being expanded to move to an expanded (or deployed) state. The plurality of struts 22 may be configured to move closer together to allow the frame 20 to move to the collapsed state. The width of the openings between the struts 22 may be reduced as the frame 20 is moved to the collapsed (or undeployed or unexpanded) state, with the length of the openings increasing. The struts 22 of the frame 20 may be configured to circumferentially move away from each other to move to the expanded state. The width of the openings between the struts 22 may be increased as the frame 20 is moved to the expanded (or deployed) state, with the length of the openings decreasing.

The body 12 may further include one or more covers 27 that may cover a portion of the frame 20. A cover 27 may comprise a skirt that may extend circumferentially about the frame 20, and may be positioned to form all or a portion of an outer surface 30 of the valve body 12. A cover 27 may enhance securement of the implant 10 when deployed at a desired location within the patient's body.

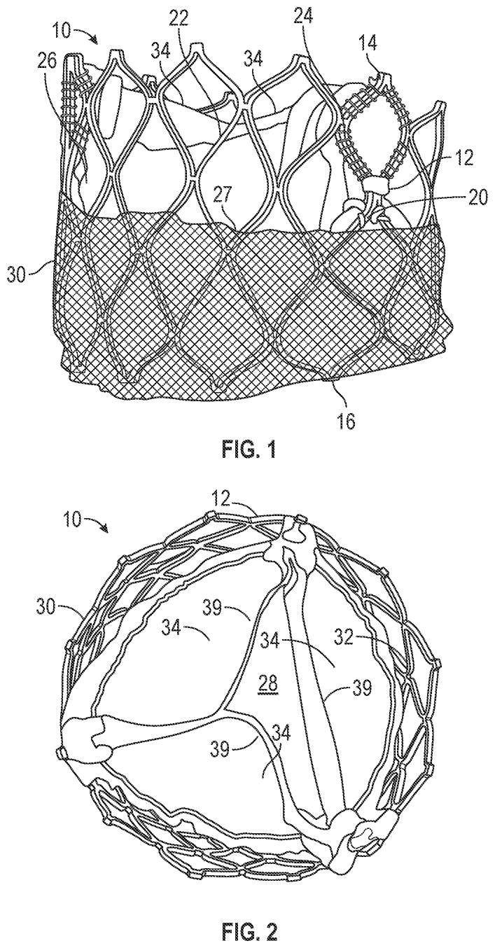

The body 12 may surround a flow channel 28 (as marked in FIG. 2) that may allow for flow of fluid (e.g., blood or another fluid) through the body 12. The body 12 may include an outer surface 30 that may face outward from the body 12 and may include an inner surface 32 (as marked in FIG. 2) that faces the flow channel 28. The outer surface 30 may comprise an anchoring surface that may be utilized to anchor the implant 10 within the desired portion of the patient's body (e.g., a heart valve annulus if desired). The outer surface 30 may apply a force radially outward to anchor the implant 10 within an annulus.

A plurality of valve leaflets 34 may be positioned within the flow channel 28 and may extend inward from the inner surface 32 of the valve body 12. Each valve leaflet 34 may include a proximal end 39 that may form an edge of the valve leaflet 34 in the outflow direction. The proximal end 39 may be opposite the distal ends of each leaflet. The implant 10 may include three valve leaflets 34 as shown in FIGS. 1 and 2, or may include a greater or lesser number of valve leaflets 34 as desired.

The valve leaflets 34 may move between an opened state (in which fluid flows through the flow channel 28) and a closed state (in which fluid flow is impeded through the flow channel 28), which may mimic the motion of native heart valve leaflets. The valve leaflets 34 may have the proximal ends 39 move towards each other in a radially inward manner and contact each other to close the valve, and then move away from each other in a radially outward manner to open the valve. The valve leaflets 34 may open in a proximal direction. FIG. 2, for example, illustrates the valve leaflets 34 in an opened state, with the valve leaflets 34 positioned away from each other in a radially outward manner. The plurality of valve leaflets 34 may be configured to open to allow for flow in the proximal or outflow direction of the prosthetic valve 10, and may be configured to close to impede flow in the distal or inflow direction of the prosthetic valve 10.

FIG. 2, for example illustrates a top schematic view of the implant 10 which may have an asymmetric shape. FIG. 2 illustrates the valve leaflets 34 in an opened configuration, allowing fluid flow through the flow channel 28.

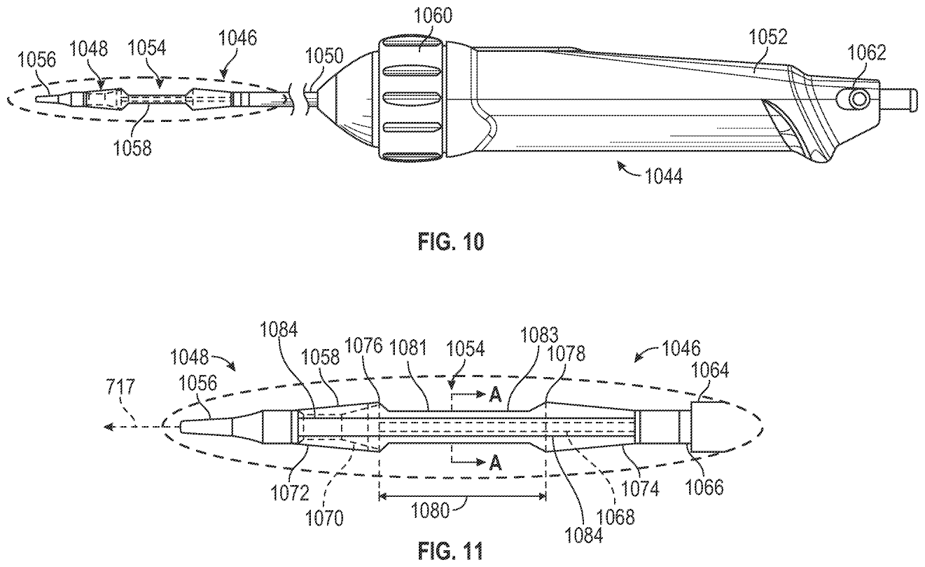

The implant 10 may be configured to be deployed to a portion of a patient's body. The implant 10 may be configured to be delivered to an implantation site utilizing a delivery apparatus. FIG. 10, for example, illustrates an embodiment of a delivery apparatus 1044 that may be utilized to deliver the implant (e.g., a prosthetic valve such as a THV as shown in FIGS. 1 and 2) to a desired implantation site. The delivery apparatus 1044 may include an elongate shaft 1046 having a distal portion 1048 and a proximal portion 1050. The proximal portion 1050 may couple to a housing in the form of a handle 1052. The distal portion 1048 may include an implant retention area 1054 and a distal tip that may include a nose cone 1056.

The distal portion 1048 may further include an inflatable body 1058 that may be in the form of a balloon.

The handle 1052 may be configured for a user to grip to operate the delivery apparatus 1044 and to maneuver the delivery apparatus 1044 through the vasculature of the patient's body. For example, the handle 1052 may be moved distally to advance the elongate shaft 1046 distally within the patient's body and may be moved proximally to retract the elongate shaft 1046 proximally within the patient's body. As such, the implant retention area 1054 and accordingly the implant may be moved and positioned with the operation of the handle 1052.

A control mechanism 1060 may further be coupled to the handle 1052. The control mechanism 1060 may be configured to be operated to bend the elongate shaft 1046 as desired. For example, one or more pull tethers may extend along the elongate shaft 1046 and operation of the control mechanism 1060 may push or pull the one or more pull tethers to cause the elongate shaft 1046 to bend. The bending of the elongate shaft 1046 accordingly may be controlled by the control mechanism 1060. As shown in FIG. 10, the control mechanism 1060 may comprise a rotatable body in the form of a control knob that may be rotated to push or pull the pull tether and cause the elongate shaft 1046 to bend. Other forms of control mechanisms may be utilized as desired.

A fluid port 1062 may further be coupled to the handle 1052 and may be utilized to transfer fluid to and from the inflatable body 1058 as desired. The configuration of the handle 1052 may be varied in other embodiments as desired.

FIG. 11 illustrates a close-up view of the distal portion 1048 of the elongate shaft 1046. The elongate shaft 1046 may include one or more shafts, which may include one or more sheaths extending over each other. For example, the elongate shaft 1046 may include an outer sheath 1064 that may be configured to extend over and be slidable relative to a mid-shaft or intermediate sheath 1066 that may comprise a shaft interior of the outer sheath 1064 (within the lumen of the outer sheath 1064). The intermediate sheath 1066 may extend over an interior shaft 1068 that may extend to the nose cone 1056 of the elongate shaft 1046. The interior shaft 1068 may be surrounded by the inflatable body 1058. The interior shaft 1068 in embodiments may include a fluid conduit that may allow fluid to be passed into and out of the inflatable body 1058 for inflating and deflating the inflatable body 1058 respectively. Other shafts may include one or more fluid conduits for inflating and deflating the inflatable body 1058 as desired.

The interior shaft 1068 may further comprise a distal shoulder 1070 that may be positioned distal of the implant retention area 1054. The distal shoulder 1070 comprises a portion of the delivery apparatus 1044 positioned distal of the implant retention area 1054, along with other portions such as the distal tip including a nose cone 1056 and a distal end 1072 of the inflatable body 1058. The distal shoulder 1070 may protrude radially outward from the interior shaft 1068 and may have a conical shape as desired. The taper of the conical shape may be configured such that the size of the distal shoulder 1070 increases in a direction towards the implant retention area 1054. The distal shoulder 1070 may be configured to protect an implant positioned within the implant retention area 1054 as the elongate shaft 1046 is advanced through the patient's body. For example, an outer diameter of the distal shoulder 1070 may be at or greater than a diameter of the implant when the implant is in a crimped state, thus shielding the leading edge (such as the distal end of the implant) from contacting a portion of the patient's body or snagging or snaring on a sheath that the elongate shaft 1046 may be advanced through. In embodiments, the configuration of the delivery apparatus may be varied from the configuration shown in FIGS. 10 and 11.

The implant retention area 1054 may be configured for the implant 10 to be crimped over the inflatable body 1058 and positioned upon a central body 1081 of the inflatable body 1058, which may be between a distal shoulder 1076 and a proximal shoulder 1078 of the inflatable body 1058. The implant 10 may be positioned proximal of the distal shoulder 1076 and crimped in such a position. In certain embodiments, the outer sheath 1064 may be advanced distally to cover the implant positioned within the implant retention area 1054 when the implant is crimped to the inflatable body 1058.

In embodiments, the interior shaft 1068 may include a shoulder (not shown) that is proximal of the implant retention area 1054. The proximal shoulder 1078 of the inflatable body 1058 may extend over the proximal shoulder of the interior shaft 1068 in such an embodiment. Notably, in embodiments, the proximal shoulder 1078 of the inflatable body 1058 may be shaped as a shoulder without extending over a shoulder of the interior shaft 1068. As such, the interior shaft 1068 may lack an interior proximal shoulder in embodiments.

The delivery apparatus 1044 may include a dilation device for the implant 10, which may be in the form of the inflatable body 1058. The inflatable body 1058 may have a distal end 1072 and a proximal end 1074, and may extend over the interior shaft 1068 and the distal shoulder 1070. A central body 1081 may be positioned between the proximal end 1074 and the distal end 1072 and may have a length 1080. The central body 1081 may be configured to press an inner surface 32 of the implant 10 (marked in FIG. 2) to dilate the implant 10.

The distal end 1072 of the inflatable body 1058 may couple to the nose cone 1056 and the proximal end 1074 may couple to the intermediate sheath 1066. The inflatable body 1058 may extend along the length of the interior shaft 1068 and may encircle the interior shaft 1068. The inflatable body 1058 is shown in a deflated state in FIG. 11, and may have a distal shoulder 1076 and a proximal shoulder 1078. The implant retention area 1054 may have a length between the distal shoulder 1076 and the proximal shoulder 1078 that may correspond to the length 1080 of the central body 1081.

The inflatable body 1058 may include an exterior surface 1083. The exterior surface 1083 may be configured to be pressed against the inner surface 32 of the implant 10 (marked in FIG. 2) to dilate the implant 10.

The inflatable body 1058 may include a plurality of folds 1084 or pleats. The folds 1084 or pleats may be present upon the inflatable body 1058 being in an uninflated state and may be present upon formation of the inflatable body 1058. The folds 1084 or pleats may allow the inflatable body 1058 to be in an uninflated, undeployed, unexpanded, or compressed configuration and then expanded radially outward to an inflated, deployed, expanded, or uncompressed configuration in which the diameter of the inflatable body 1058 increases and the implant 10 is expanded radially outward to be deployed.

In operation, the inflatable body 1058 may be formed with folds 1084 or pleats and coupled to the delivery apparatus 1044. The inflatable body 1058 may be provided in the uninflated, undeployed, unexpanded, or compressed configuration. The implant 10 may then be positioned over the inflatable body 1058 and may be crimped or otherwise compressed to the inflatable body 1058. The delivery apparatus 1044 may then be inserted into the patient's body. With the implant 10 in a desired position within the patient's body, for example proximate an implantation site, fluid may be flowed through the fluid port 1062, for example, to inflate the inflatable body 1058. The inflatable body 1058 may be expanded radially outward to the inflated, deployed, expanded, or uncompressed configuration in which the diameter of the inflatable body 1058 increases and the implant 10 is expanded radially outward to be deployed.

As discussed herein, providing the implant 10 in an asymmetric shape upon deployment of the implant 10 may be undesirable. Referring to FIG. 1, the height of the implant 10, for example, may be asymmetrical, with the height of the rightmost portion of the implant 10 shown in FIG. 1 being less than the height of the leftmost portion of the implant 10 shown in FIG. 1. This generally undesired feature may be produced for a variety of reasons. For example, the expansion of an inflatable body used to expand the implant 10 may be asymmetrical. The asymmetrical expansion may result from a certain portion of the inflatable body expanding or unfurling at a different time than another portion, resulting in an asymmetrical expansion of the inflatable body. As such, the asymmetrically expanded inflatable body may expand different portions of the implant 10 at different rates, for example, the leftmost portion of the implant 10 shown in FIG. 1 may expand earlier than the rightmost portion of the implant 10 shown in FIG. 1, which may cause the height asymmetry shown in FIG. 1. Other features may result in an undesired asymmetrical deployment of the implant 10.

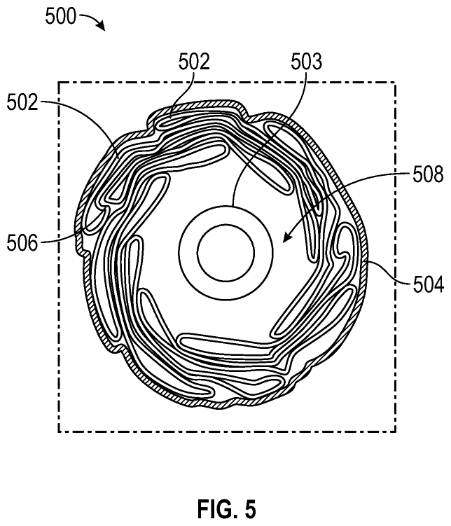

Features of an inflatable body may increase the possibility of an undesired asymmetrical deployment of the implant 10. FIG. 5, for example, illustrates features of an inflatable body that may be undesirable. The view of FIG. 5 is a cross sectional view of an inflatable body 500 taken along a mid-line extending perpendicular to the axis of the inflatable body 500. Folds 502 or pleats of the inflatable body 500 are shown overlapping each other. A sheath 504 is shown extending over the folds 502 or pleats, however, in practice the sheath 504 may be excluded. An interior shaft 503 or guidewire shaft is shown surrounded by the inflatable body 500.

The configuration of the inflatable body 500 may yield high deployed implant height asymmetry. Features of the inflatable body 500 that may contribute to such asymmetry for the implant 10 may include tight compact inflatable body folds or pleats. Such features may include disorganized inflatable body folds or pleats. Such features may include tight hooked inflatable body folds or pleats. Tight, compact inflatable body folds or pleats, disorganized inflatable body folds or pleats, and/or tight hooked inflatable body folds or pleats may lead to a high deployed implant height asymmetry. For example, for tight, compact inflatable body folds or pleats, disorganized inflatable body folds or pleats, and/or tight hooked inflatable body folds or pleats, one or more of the folds or pleats may catch during deployment. Accordingly, the inflatable body for any of the tight, compact inflatable body folds or pleats, disorganized inflatable body folds or pleats, or tight hooked inflatable body folds or pleats may be deployed in an asymmetric manner due to the one or more folds or pleats catching (e.g., sticking together) and therefore failing to deploy at the same rate as other pleats of the particular inflatable body (e.g., tight, compact inflatable body folds or pleats, disorganized inflatable body folds or pleats, and/or tight hooked inflatable body folds or pleats).

FIG. 5, for example, illustrates a hook 506 of a fold 502 or pleat for the inflatable body 500. Such a hook 506 may catch and open at the end of deployment at a different timing than the remainder of the folds 502. The folds 502 may thus unfurl in an asymmetric manner due to the presence of the hook 506. With regard to the implant 10, such asymmetry may thus be developed in the frame 20 and may continue through to full deployment, which may undesirably result in an asymmetric deployment as shown in FIG. 1.

Further, tight compact inflatable body folds or pleats or disorganized inflatable body folds or pleats may result from a desire for a large inside diameter of an interior lumen 508 for fluid flow and a small outside diameter of the inflatable body 500 to minimize the diameter of the crimped implant 10. However, such tight compact inflatable body pleats or disorganized inflatable body folds or pleats may produce an asymmetrical unfurlment of the folds or pleats during expansion of the inflatable body, thus producing an asymmetric expansion of the inflatable body and the implant.

Systems, apparatuses, and methods described herein may be used to lessen or eliminate such an undesired asymmetric resulting shape for the implant 10 in some cases. Features of inflatable bodies disclosed herein may be designed to reduce or eliminate undesired features disclosed in regard to FIG. 5 and as shown in an asymmetric deployment in FIGS. 1 and 2.

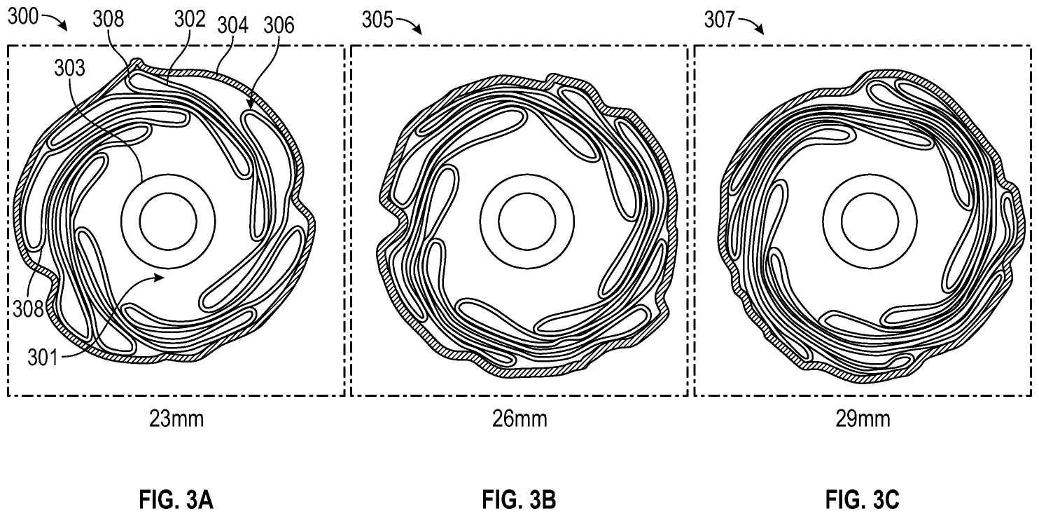

FIG. 3A, for example, illustrates a cross sectional view of a dilation device in the form of an inflatable body 300 that may be utilized according to embodiments herein. The cross sectional view is taken along a mid-line extending perpendicular to the axis of the inflatable body 300. For example, the mid-line may correspond to the line A-A shown in FIG. 11. Folds 302 or pleats of the inflatable body 300 are shown overlapping each other. A sheath 304 is shown extending over the folds 302 or pleats, however, in practice the sheath 304 may be excluded. An interior shaft 303 or guidewire shaft is shown surrounded by the inflatable body 300 and positioned within an interior lumen 301 of the inflatable body 300. The inflatable body 300 may include an exterior surface 306 that may be configured to be pressed against the inner surface 32 of the implant 10 (marked in FIG. 2) to dilate the implant 10, with the sheath 304 removed.

Features of the inflatable body 300 may correspond to features of the inflatable body 1058 shown in FIG. 11. For example, the inflatable body 300 may be configured similarly as the inflatable body 1058 shown in FIG. 11. The folds 302 or pleats may correspond to the folds 1084 or pleats shown in FIG. 11. The interior shaft 303 may correspond to the interior shaft 1068 shown in FIG. 11. The inflatable body 300 may be coupled to a delivery apparatus and inflated or deflated in a similar manner as the inflatable body 1058 shown in FIG. 11. The inflatable body 300, similar to the inflatable body 1058 shown in FIG. 11 may have a proximal end and a distal end, and a central body positioned between the proximal end and the distal end and having a length. The central body may be configured to press an inner surface of the implant to dilate the implant.

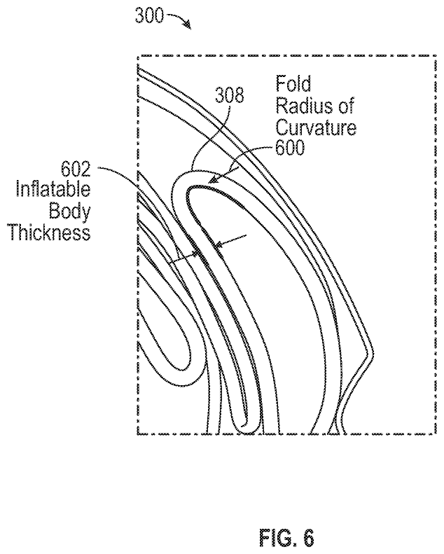

Features of the inflatable body 300 may reduce the possibility of asymmetric deployment of an implant, or may provide other beneficial results. Such features may include the configuration of one or more outer inflatable body fold 308 radius of curvature of the inflatable body 300. The outer inflatable body fold 308 radius of curvature may be configured to be greater than or equal to one half a thickness of the inflatable body 300. FIG. 6, for example, illustrates a close up view of an outer inflatable body fold 308 of the inflatable body 300. The radius of curvature 600 of the outer inflatable body fold 308, relative to the inflatable body thickness 602 is shown to be greater than or equal to one half the thickness 602 of the inflatable body 300.

The outer inflatable body fold 308 radius of curvature 600 may be greater than or equal to one half a thickness 602 of the inflatable body 300 to provide a variety of benefits, which may include reducing asymmetric deployment of an implant positioned upon the exterior surface 306 of the inflatable body 300. The outer inflatable body fold 308 radius of curvature 600 may be provided at greater than or equal to one half a thickness 602 of the inflatable body 300 during the formation of the inflatable body 300, which may include a pleat and fold operation upon the inflatable body 300, which the folds are formed, and/or during a compression operation applied to the pleated and folded inflatable body 300 that may compress the inflatable body 300. For example, the pleat and fold operation and/or the compression operation may be applied to produce the outer inflatable body fold 308 radius of curvature 600 being provided at greater than or equal to one half a thickness 602 of the inflatable body 300 due to the force of compression or configuration of the pleat and fold operation, among other factors during formation. The outer inflatable body fold 308 radius of curvature 600 may be greater than or equal to one half a thickness 602 of the inflatable body 300 upon insertion of the inflatable body 300 into the patient's body and upon deployment of an implant positioned upon the exterior surface 306 of the inflatable body 300.

The outer inflatable body fold 308 radius of curvature 600 being greater than or equal to one half a thickness 602 of the inflatable body 300 may reduce the possibility of creasing of the folds, which may reduce the possibility of uneven unfurling of the folds during expansion of the inflatable body 300. Creases, for example, may cause uneven unfurling of the folds as the creases may reduce the possibility of a rolling movement of the folds during expansion of the inflatable body, and may increase the possibility of an undesired sliding movement of the folds. Further, the outer inflatable body fold 308 radius of curvature 600 being greater than or equal to one half a thickness 602 of the inflatable body 300 may reduce the possibility of hooks, such as a hook 506 shown in FIG. 5, which may be undesired. Further, the outer inflatable body fold 308 radius of curvature 600 being greater than or equal to one half a thickness 602 of the inflatable body 300 may enhance the ability of inflation fluid to pass within the interior of the folds, to allow for ease of symmetrical expansion of the inflatable body 300. Various other benefits may be provided.

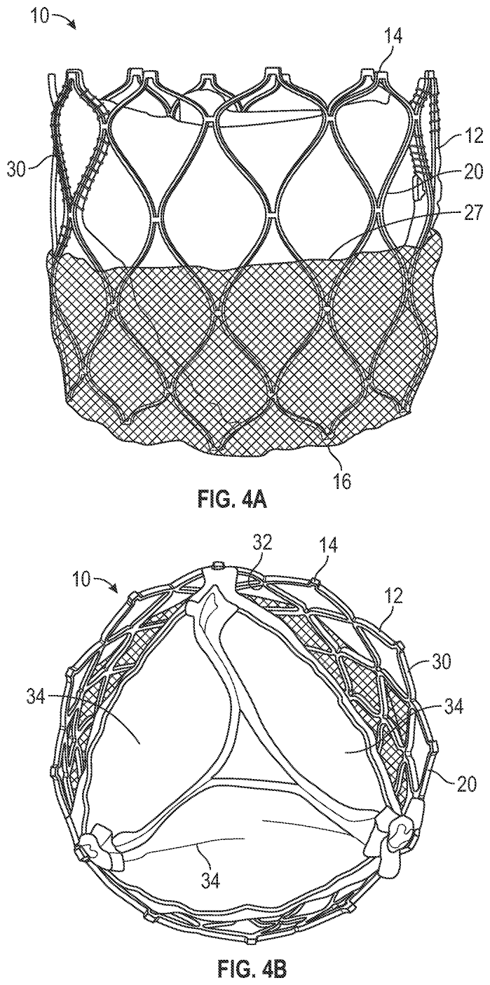

A resulting implant 10 may have a greater possibility of a symmetric shape, including height, upon deployment. FIGS. 4A and 4B, for example, illustrates a shape of the implant 10 that may be deployed with the inflatable body 300. The body 12 may have a symmetrical cylindrical shape as shown in FIGS. 4A and 4B. The cylindrical shape may include a uniform outer diameter for the outer surface 30 of the body 12 in embodiments, and may include a uniform inner diameter of the inner surface 32. A reduced possibility of paravalvular leakage and improper coaptation of the leaflets 34 may be provided.

In embodiments, the configuration of the implant may be varied. The implant may have another shape (e.g., a tapered or “V” shape, or a bulb shape, or other desired shape), yet a symmetrical deployment may be provided. Other configurations of implant may be utilized as desired.

Referring back to FIG. 6, other outer inflatable body fold radius of curvatures may be provided in embodiments. In embodiments, the outer inflatable body fold 308 radius of curvature 600 may be greater than the thickness 602 of the inflatable body 300. In embodiments, the outer inflatable body fold 308 radius of curvature 600 may be greater than two times the thickness 602 of the inflatable body 300. In embodiments, the outer inflatable body fold 308 radius of curvature 600 may be between one and five times the thickness 602 of the inflatable body 300. In embodiments, the outer inflatable body fold 308 radius of curvature 600 may be between one half the thickness 602 of the inflatable body 300 and the thickness 602 of the inflatable body 300. The configurations may each provide a reduction in asymmetries in implant height after deployment due to an enhanced ease with which the folds may unfurl. Such configurations may reduce the tightness of the folds, rigidity of the folds, and may reduce hooking of the folds. Such configurations may comprise loose loops of the inflatable body that may provide such beneficial results.

In embodiments, at least one of the outer inflatable body fold radius of curvatures may be configured in such a manner. In embodiments, multiple of the outer inflatable body fold radius of curvatures may be configured in such a manner.

FIG. 3B illustrates a larger sized inflatable body 305 that may include such outer inflatable body fold radius of curvatures. FIG. 3C illustrates a further larger sized inflatable body 307 that may include such outer inflatable body fold radius of curvatures. The inflatable body 300 in FIG. 3A, for example, may be configured to deploy a 23 millimeter implant, the inflatable body 305 in FIG. 3B may be configured to deploy a 26 millimeter implant, and the inflatable body 307 in FIG. 3C may be configured to deploy a 29 millimeter implant. Other sizes may be utilized in embodiments as desired.

Other features may be utilized in embodiments herein. For example, referring to FIG. 3A, in embodiments, a lubricious exterior surface 306 may be provided for the inflatable body 300. The lubricious exterior surface 306 may be utilized in combination with, or in lieu of, the configurations of the outer inflatable body fold radius of curvatures discussed in regard to the inflatable body 300. The lubricious exterior surface 306 may be provided to reduce friction between the folds of the inflatable body 300, to enhance the ability of the folds to unfurl during inflation of the inflatable body 300. For example, enhanced rolling of the folds may be provided due to reduced friction of the folds with each other.

In embodiments, the lubricious exterior surface 306 may have a variety of forms. For example, in embodiments, the lubricious exterior surface 306 may comprise a lubricous coating on the inflatable body 300. In embodiments, the lubricious coating of the exterior surface 306 may be a biocompatible material. Examples of biocompatible lubricious coatings include, but are not limited to silicone-based lubricant, a hydrophilic polymer such as polyvinylpyrrolidone (PVP), PVP/vinyl acetate copolymer, or polyethylene oxide, or other known or yet to be developed biocompatible lubricious substances. The lubricious coating, for example, may be a fluid coating that is applied to the exterior surface of the inflatable body 300. The lubricious coating may be swabbed upon the exterior surface of the inflatable body 300 between the folds of the inflatable body 300 or in another manner as desired.

In embodiments, the lubricious exterior surface 306 may comprise a surface thermally bonded to the inflatable body. For example, the thermally bonded surface may be a low friction surface that is provided as the exterior surface of the inflatable body 300. In embodiments, the lubricious exterior surface 306 may comprise a textured surface. For example, a lubricious surface may be generated by texturing a surface for low-friction applications by making it rougher (stochastic) and then remove any protruding edges. In embodiments, a textured surface may be provided in a molding process of the inflatable body 300, in which the mold for the inflatable body 300 may include the texture that is applied to the exterior surface during molding of the inflatable body. Another manner to generate a low friction surface may be to use porous materials where the pores act as reservoirs for lubricants. In such an example, new pores may appear as the material is worn. In embodiments, a lubricous exterior surface may comprise a bead blasted surface, which may be utilized to provide a textured surface.

The lubricious exterior surface may be utilized to enhance the ability of the folds of the inflatable body 300 to unfurl relative to each other, and may reduce the possibility of sticking to each other. The lubricious exterior surface in embodiments may be positioned between the folds to allow for enhanced unfurling. Embodiments such as the inflatable bodies 305, 307 shown in FIGS. 3B and 3C may include use of a lubricious exterior surface or other inflatable bodies as disclosed herein. The lubricious exterior surface may be utilized solely or in combination with any other features disclosed herein.

Other features that may be utilized in embodiments herein may include a relation between a pleat height and a pleated inside diameter of an inflatable body. Such a relation may be formed during a pleat and fold operation of the inflatable body.

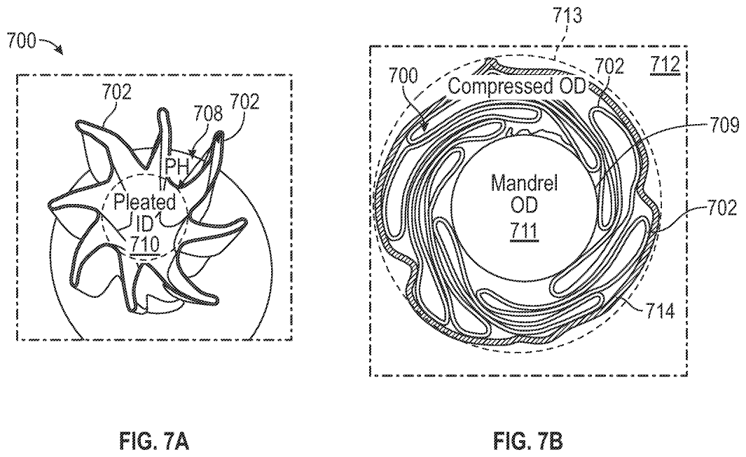

Referring to FIG. 7A, during formation of an inflatable body 700, the inflatable body 700 may be pleated, to form the configuration shown in FIG. 7A. Such a pleating operation may form the radially extending pleats 702 shown in FIG. 7A. For example, jaws of a tool may press into the inflatable body 700 to deflect and form the pleats 702 shown in FIG. 7A. The tool may be configured to produce a resulting relationship between the pleat height (PH) (708) and the pleated inside diameter (ID) 710 (the diameter formed by the innermost portions of the pleats 702). The tool, for example, may be configured to have a depth of the jaws set to form a resulting relationship between the pleat height 708 and the pleated inside diameter 710.

In embodiments, the relationship of the pleat height 708 and the pleated inside diameter 710 may be formed such that the pleated inside diameter 710 is greater than the pleat height 708. Such a relationship may increase the outer inflatable body fold radius of curvature, and may loosen the tightness of the pleats. Reduced possibility of hooks of the pleats (as shown as hook 506 in FIG. 5 for example) may result. For example, in an embodiment, the pleated inside diameter 710 may be about 3.9 millimeters, and the pleat height 708 may be less than this pleated inside diameter 710, although other pleated inside diameters 710 may be utilized in embodiments. The relationship between the pleat height 708 and the pleated inside diameter 710 may remain upon a compression operation compressing the pleats 702 shown in FIG. 7A to a lowered configuration as shown in FIGS. 3A-3C and 7B for example.

The relation between a pleat height and a pleated inside diameter of an inflatable body may be utilized solely or in combination with any other features disclosed herein.

Other features that may be utilized in embodiments herein may include a relation between a compressed outer diameter of the inflatable body and an outer diameter of a mandrel positioned within the inflatable body. Such a relation may be formed during a compression operation applied to the inflatable body. For example, following a pleat and fold operation of the inflatable body as shown in FIG. 7A, the pleats may be compressed to form a resulting lowered configuration as shown in FIG. 7B, and FIGS. 3A-3C for example. In embodiments, a compression operation may be otherwise performed upon the inflatable body.

In a compression operation, a mandrel 709 may be inserted into the interior lumen of the inflatable body 700. The inflatable body 700 may be formed using a mandrel 709 positioned within the inflatable body 700. A compression tool 712 (positioned exterior of the inflatable body 700) may be utilized to apply a radially inward force to the inflatable body 700 to compress and lower the pleats 702 to an amount relative to the mandrel 709. In embodiments herein, a ratio of a compressed outer diameter 713 of the inflatable body 700 to an outer diameter 711 of the mandrel 709 may be 2.5 or larger. As such, the compression tool 712 may compress the pleats 702 to a distance from the outer diameter 711 of the mandrel 709 that corresponds to a ratio of a compressed outer diameter 713 of the inflatable body 700 to an outer diameter 711 of the mandrel 709 that may be 2.5 or larger. The relationship of the outer diameter 711 of the mandrel 709 to the compressed outer diameter 713 of the inflatable body 700 may be set to loosen the tightness of the pleats, and reduced possibility of hooks of the pleats (as shown as hook 506 in FIG. 5 for example) may result. For example, a compressed outer diameter 713 of the inflatable body 700 may be 3.9 millimeters in embodiments, with an outer diameter 711 of the mandrel 709 being 1.5 millimeters in embodiments. In embodiments, other sizes and relationships may be utilized as desired.

In embodiments, a sheath 714 positioned around the inflatable body 700 during a compression operation may influence pleat symmetry. The sheath 714 may comprise a shrink tubing or another form of tubing that may have an interior diameter. A smaller starting interior diameter may yield more organized pleats. When a starting interior diameter of shrink tubing is too large, disorganized pleats may result, however. The sheath 714 may be removed following the compression operation and may be removed prior to use of the inflatable body 700. The mandrel 709 further may be removed prior to use of the inflatable body 700 and a lumen for inflating the inflatable body 700 may be inserted in its place, among other features.

The relation between a compressed outer diameter 713 of the inflatable body 700 to an outer diameter 711 of the mandrel 709 may be utilized solely or in combination with any other features disclosed herein. For example, a lubricious coating as disclosed herein, or a relationship between a pleat height and a pleated inside diameter as disclosed herein may be utilized. In embodiments, a configuration of an outer inflatable body fold radius of curvature as disclosed herein may be utilized, which may be predetermined for formation of the inflatable body.

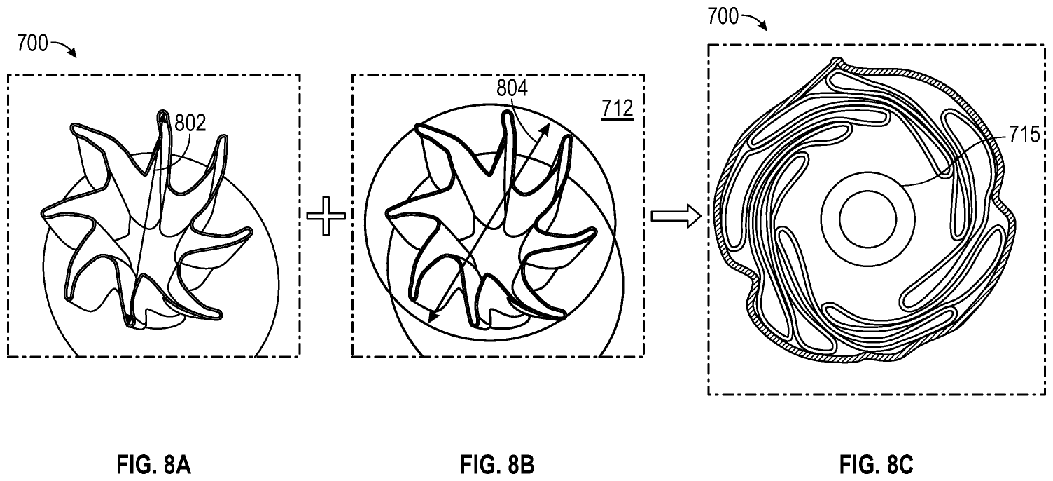

Other features that may be utilized in embodiments herein may include a relationship between an interior diameter 804 of the compression tool 712 and the outer pleated diameter 802 of the pleated inflatable body 700. For example, referring to FIG. 8A, a pleated inflatable body 700 may have an outer pleated diameter 802. Referring to FIG. 8B, the pleated inflatable body 700 may be inserted into a compression tool 712 having an interior diameter 804 that is larger than the outer pleated diameter 802 of the pleated inflatable body 700. Thus, the compression operation may reduce the possibility of hooks forming with the pleats as shown in FIG. 8C for example. The relation between the interior diameter 804 of the compression tool 712 and the outer pleated diameter 802 of the pleated inflatable body 700 may be utilized solely or in combination with any other features disclosed herein.

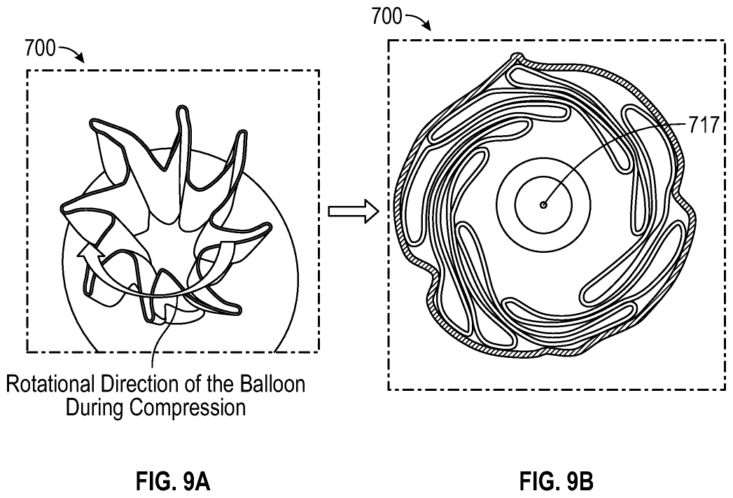

Other features that may be utilized in embodiments herein may include rotating the inflatable body 700 along an axis from the proximal end of the inflatable body 700 through the distal end of the inflatable body 700 while compressing the inflatable body 700. The axis may comprise a longitudinal axis 717 as marked in FIG. 9B and FIG. 11. For example, referring to FIG. 9A, the pleated inflatable body 700 may be rotated while a compression tool 712 (as marked in FIG. 7B for example) compresses the pleated inflatable body 700. The pleated inflatable body 700 may be rotated in a direction, as illustrated, and the rotation may be relative to the compression tool 712. By rotating the inflatable body during compression, a reduced possibility of hooks forming with the pleats may result, as shown in FIG. 9B for example. For example, rotation of the inflatable body during compression may lead to a looser compression of the inflatable body and reduction of hooks forming. The rotation of the inflatable body may be utilized solely or in combination with any other features disclosed herein.

The inflatable body 700 may otherwise correspond to the inflatable body 1058 shown in FIG. 11. For example, the inflatable body 700 may be configured similarly as the inflatable body 1058 shown in FIG. 11. The folds or pleats of the inflatable body 700 may correspond to the folds 1084 or pleats shown in FIG. 11. An interior shaft 715 shown in FIG. 8C may correspond to the interior shaft 1068 shown in FIG. 11. The inflatable body 700 may be coupled to a delivery apparatus and inflated or deflated in a similar manner as the inflatable body 1058 shown in FIG. 11. The inflatable body 700, similar to the inflatable body 1058 shown in FIG. 11 may have a proximal end and a distal end, and a central body positioned between the proximal end and the distal end and having a length. The central body may be configured to press an inner surface of the implant to dilate the implant.

In operation, the formed inflatable body may be coupled to a delivery apparatus and may comprise a dilation device for an implant. The dilation device may be inserted into a body. An implant may be positioned upon the dilation device prior to entry to the patient's body or may be positioned upon the dilation device within the patient's body.

The implant may be dilated utilizing the dilation device and deployed to the desired portion of the patient's body. For example, with reference to FIGS. 10 and 11, with the implant 10 in a desired position within the patient's body, for example proximate an implantation site, fluid may be flowed through the fluid port 1062, for example, to inflate the inflatable body. The inflatable body may be expanded radially outward to the inflated, deployed, expanded, or uncompressed configuration in which the diameter of the inflatable body increases and the implant 10 is expanded radially outward to be deployed. The dilation device may be configured according to any of the embodiments of dilation devices disclosed herein. Other methods may be utilized according to embodiments herein.

The implant may comprise a prosthetic heart valve and may be deployed to a heart valve. The delivery apparatus, for example, may be advanced through the vasculature of the patient's body to approach the heart valve. The delivery apparatus may be deflected via a control mechanism or other device to direct the delivery apparatus to the implantation site. The dilation device may be expanded or inflated to deploy the implant to the implantation site.

In an embodiment in which the implant is to be deployed to an aortic valve, the delivery apparatus may approach the native aortic valve via the aortic arch. Other approaches (a ventricular approach) may be utilized as desired. An elongate shaft of the delivery apparatus may pass through the vasculature, with a handle remaining exterior to the patient's body. The implant in the form of a prosthetic aortic valve may be deployed to the aortic valve. Other implantation sites may be utilized, such as a mitral, pulmonary, or tricuspid valve. Other implantation sites within the heart or within the patient's body may be utilized. Various forms of implants may be utilized, which may comprise medical implants for treatment of a patient's body, and may comprise prosthetic heart valves or stents or other forms of implants.

Features of embodiments may be modified, substituted, excluded, or combined.

In addition, the methods herein are not limited to the methods specifically described, and may include methods of utilizing the systems and apparatuses disclosed herein.

The steps of the method may be modified, excluded, or added to, with systems, apparatuses, and methods disclosed herein.

The features of the embodiments disclosed herein may be implemented independently of the delivery apparatuses, or independent of other components disclosed herein. The various apparatuses of the system may be implemented independently.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A dilation device for an implant, the dilation device comprising:
an inflatable body having a proximal end and a distal end, and a central body positioned between the proximal end and the distal end and having a length, the central body configured to press an inner surface of the implant to dilate the implant, the inflatable body including at least one outer inflatable body fold radius of curvature greater than or equal to one half a thickness of the inflatable body.

2. The dilation device of claim 1, wherein the at least one outer inflatable body fold radius of curvature is greater than the thickness of the inflatable body.

3. The dilation device of claim 1, wherein the at least one outer inflatable body fold radius of curvature is greater than two times the thickness of the inflatable body.

4. The dilation device of claim 1, wherein the at least one outer inflatable body fold radius of curvature is between one and five times the thickness of the inflatable body.

5. The dilation device of claim 1, wherein the at least one outer inflatable body fold radius of curvature is between one half the thickness of the inflatable body and the thickness of the inflatable body.

6. The dilation device of claim 1, wherein the inflatable body includes an interior lumen for fluid flow.

7. The dilation device of claim 1, wherein the inflatable body includes a plurality of folds.

8. The dilation device of claim 7, wherein the plurality of folds are configured to unfurl during inflation of the inflatable body.

9. The dilation device of claim 1, wherein a diameter of the inflatable body increases upon inflation of the inflatable body.

10. The dilation device of claim 1, wherein the inflatable body includes a distal shoulder.

11. A dilation device for an implant, the dilation device comprising:
an inflatable body having a proximal end and a distal end, and a central body positioned between the proximal end and the distal end and having a length, the central body configured to press an inner surface of the implant to dilate the implant, the inflatable body including a lubricious exterior surface.

12. The dilation device of claim 11, wherein the lubricious exterior surface comprises a lubricious coating on the inflatable body.

13. The dilation device of claim 12, wherein the lubricious coating comprises a biocompatible material.

14. The dilation device of claim 13, wherein the biocompatible material comprises a silicone-based lubricant.

15. The dilation device of claim 11, wherein the lubricious exterior surface comprises a surface thermally bonded to the inflatable body.

16. The dilation device of claim 11, wherein the lubricious exterior surface comprises a textured surface.

17. An implant delivery system comprising:
a delivery apparatus; and
an inflatable body coupled to the delivery apparatus and having a proximal end and a distal end, and a central body positioned between the proximal end and the distal end and having a length, the central body configured to press an inner surface of an implant to dilate the implant, the inflatable body including at least one outer inflatable body fold radius of curvature greater than or equal to one half a thickness of the inflatable body.

18. The implant delivery system of claim 17, wherein the at least one outer inflatable body fold radius of curvature is greater than the thickness of the inflatable body.

19. The implant delivery system of claim 17, wherein the at least one outer inflatable body fold radius of curvature is greater than two times the thickness of the inflatable body.

20. The implant delivery system of claim 17, wherein the at least one outer inflatable body fold radius of curvature is between one and five times the thickness of the inflatable body.
